(19) United States
(12) Patent Application Publication
(10) Pub. No.: US 2006/0009629 A1
(43) Pub. Date: Jan. 12, 2006

(54) Organic light emitting device material and organic light emitting devcie

(76) Inventors: Yoshiaki Takahashi, Chiba (JP); Koro Shirane, Chiba (JP); Kunio Kondo, Chiba (JP)

Correspondence Address: SUGHRUE MION, PLLC, 2100 PENNSYLVANIA AVENUE, N.W., SUITE 800, WASHINGTON, DC, 20037, US

(21) Appl. No.: 10/534,761

(22) PCT Filed: Nov. 14, 2003
(86) PCT No.: PCT/JP03/14514

§ 371 Date: May 13, 2005

Related U.S. Application Data
(60) Provisional application No. 60/427,950, filed on Nov. 21, 2002.

(30) Foreign Application Priority Data
Nov. 15, 2002 (JP) 2002331555

(51) Int. Cl. C07H 21/02

(52) U.S. Cl. 536/23.1

(57) ABSTRACT

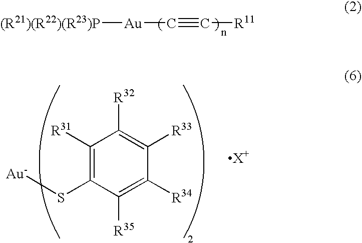

The present invention provides phosphorescent materials and polymer phosphorescent materials that generate lights of various colors including blue, green, yellow, orange and red, which are useful for high performance multicolor organic light-emitting EL devices, and also provides an organic light emitting device material containing a gold complex represented by formulae such as (2) and (6) below (symbols in the formulae are as described in the specification) in which gold is bonded to at least one atom selected from carbon, oxygen and sulfur, and organic light-emitting EL device including the material in its light-emitting layer.

### CROSS-REFERENCE TO THE RELATED APPLICATIONS

This is an application filed pursuant to 35 U.S.C. Section 111(a) with claiming the benefit of U.S. Provisional application Ser. No. 60/427,950 filed Nov. 21, 2002, under the provision of 35 U.S.C. Section 111(b), pursuant to 35 U.S.C. Section 119(e) (1).

### TECHNICAL FIELD

The present invention relates to an organic light emitting device (hereinafter, “OLED”) that emits light with electric energy and is usable for a flat display panel and a backlight used therein, an illumination light source, electrophotography, an optical device light source, a sign plate and so on and to a luminous material used therefore.

### BACKGROUND ART

Since high luminance light emission in organic light emitting devices has been demonstrated by C. W. Tang of Kodak Company in 1987 (see Appl. Phys. Lett., vol. 51, p. 913, 1987), development of materials and improvement of the structure of device have been advanced rapidly. Recently, organic light emitting devices have gone into practical use firstly in displays for car audio sets, cellular phones and the like. Currently, to put such organic electroluminescent (EL) devices to a wider range of uses, development of materials for improving the light emission efficiency and durability, or development for applying them to full color displays are being actively made.

As for light emission efficiency, light emission of existing light emitting materials is fluorescence which is defined as luminescence from a singlet excited state. According to the description at page 58 of “Monthly Display”, October 1998, Separate Volume, “Organic EL Display”, the upper limit of the internal quantum efficiency of light emission in an organic EL is 25%, which is considered on the ground that the ratio of a singlet excited state to a triplet excited state generated by electric excitation is 1:3.

In contrast thereto, by using an iridium complex which emits phosphorescence from a triplet excited state, M. A. Baldo et al. showed that it is possible to obtain an external quantum efficiency of 7.5%, which, assuming that the light out-coupling efficiency is 20%, corresponds to an internal quantum efficiency of 37.5% and is higher than the uppermost value 25% of quantum efficiency in case of using a fluorescent dye (Appl. Phys. Lett., Vol. 75, page 4 (1999), WO00/70655).

On the other hand, concerning luminous color, the luminous color of iridium complex Baldo et al. reported was green. Recently, while development and research on full-color displays utilizing organic EL devices and white light sources have been being performed vigorously, development of light-emitting materials that generate other colors at high efficiencies has been demanded.

Concerning the fabrication method of organic EL devices, conventionally a vacuum deposition method has been used. However, the vacuum deposition method involves problems that such a method requires a vacuum equipment and that the larger the panel using the EL device, the more difficult it becomes to form an organic thin film with a uniform thickness.

In contrast thereto, an inkjet method and a printing method, which have been developed as a film forming technique by coating, can form films under atmospheric pressure and are excellent in adaptability to fabrication of large-area devices and mass-production of devices. Since in film formation by these methods, low molecular compounds, which have the potential to cause phase separation or segregation, cannot be used, development of high molecular weight luminous materials that do not crystallize has been necessary.

As described above, it has heretofore been desired to develop phosphorescent materials and high molecular weight phosphorescent materials that generate light with a variety of luminous colors, which colors are required for high-performance multicolor organic EL devices.

### DISCLOSURE OF THE INVENTION

It is an object of the present invention to provide a phosphorescent material useful for high-performance multicolor organic EL devices.

As a result of extensive studies, the inventors of the present invention have achieved the object by the following means. That is, the present invention relates to light-emitting materials using a gold complex having a phosphorescent property, which are useful as light-emitting materials for organic EL devices and to a light-emitting device using the light-emitting materials.
1. A light-emitting material for organic light emitting device, comprising a gold complex in which gold is bonded to at least one atom selected from the group consisting of carbon, oxygen and sulfur.
2. The light-emitting material for organic light-emitting device comprising the gold complex as described in 1 above, wherein the at least one atom selected from the group consisting of carbon, oxygen and sulfur, bonded to gold is bonded to only one other atom than gold.
3. The light-emitting material for organic light-emitting device comprising the gold complex as described in 1 above, wherein the at least one atom selected from the group consisting of carbon, oxygen and sulfur, is carbon.
4. The light-emitting material for organic light-emitting device comprising the gold complex as described in 3 above, wherein the carbon atom bonded to gold is bonded to a nonmetallic element through a triple bond and to the gold through a single bond.
5. The light-emitting material for organic light-emitting device comprising the gold complex as described in 4 above, wherein the nonmetallic element is carbon.
6. The light-emitting material for organic light-emitting device as described in 5 above, wherein the gold complex is a compound represented by formula (1) 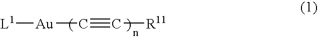
   wherein L¹ represents a monodentate ligand or a bidentate ligand, n is an integer of 1 to 5, and R¹¹ represents a hydrogen atom, a halogen atom, a cyano group, a silyl group, or an alkyl group, aryl group, alkoxy group, acyl group, carboxyl group or alkoxycarbonyl group that optionally has a heteroatom.
7. The light-emitting material for organic light-emitting device as described in 6 above, wherein the gold complex is a compound represented by formula (2) 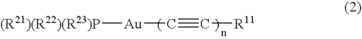
   wherein n and R¹¹ have the same meanings as described in 6 above, R²¹ to R²³ independently represent each a hydrogen atom, an amino group, a cyano group, a silyl group, or an alkyl group, aryl group, alkoxy group, aryloxy group, or alkylamino group that optionally has a heteroatom.
8. The light-emitting material for organic light-emitting device as described in 5 above, wherein the gold complex is a compound represented by formula (3) 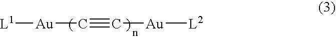
   wherein L¹ and L² independently represent each a monodentate ligand or a bidentate ligand, and n represents an integer of 1 to 5.]

9. The material for organic light emitting as described in 5 above, wherein the gold complex is a compound represented by formula (4) 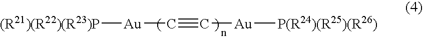
wherein n represents an integer of 1 to 5, and R²¹ to R²⁶ independently represent each a hydrogen atom, an amino group, a cyano group, a silyl group, or an alkyl group, aryl group, alkoxy group, aryloxy group or alkylamino group that optionally has a heteroatom.
10. The light-emitting material for organic light-emitting device comprising the gold complex as described in 4 above, wherein the nonmetallic element is nitrogen.
11. The light-emitting material for organic light-emitting device comprising the gold complex as described in 10 above, wherein the nitrogen atom forming a triple bond with the carbon atom bonded to gold is further bonded to another carbon atom.
12. The light-emitting material for organic light-emitting device as described in 11 above, wherein the gold complex is a compound represented by formula (5) 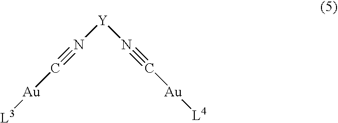
   wherein Y is an alkylene, a cycloalkylene, an arylene, or an organic group consisting of two or more of the three groups, same or different, alternately bonded to each other, and L³ and L⁴ independently represent each a monodentate or bidentate ligand.
13. The light-emitting material for organic light-emitting device comprising the gold complex as described in 1 above, wherein the at least one atom selected from the group consisting of carbon, oxygen and sulfur is sulfur.
14. The light-emitting material for organic light-emitting device comprising the gold complex as described in 13 above, wherein the gold complex is a compound represented by formula (6) 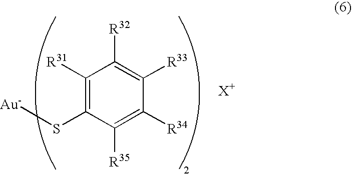
   wherein R³¹ to R³⁵ independently represent each a hydrogen atom, a halogen atom, a hydroxyl group, a nitro group, an amino group, a cyano group, a mercapto group, a silyl group, a sulfonic acid group, a sulfonic acid ester group, a phosphoric acid group, a phosphonic acid group, or an alkyl group, aryl group, alkoxy group, acyl group, a carboxyl group, an alkoxycarbonyl group or acyloxy group that optionally has a heteroatom, and X⁺ represents a monovalent cation.
15. The light-emitting material for organic light-emitting device comprising the gold complex as described in 13 above, wherein the gold complex is a compound represented by formula (7) 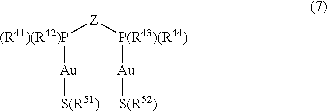
   wherein R⁴¹ to R⁴⁴, R⁵¹ and R⁵² independently represent each a hydrogen atom, a cyano group, a silyl group, or an alkyl group, aryl group or acyl group that optionally has a heteroatom, and Z represents an alkylene, a cycloalkylene, an arylene, or an organic group consisting of two or more of the three groups, same or different, alternately bonded to each other.
16. An organic light emitting device, comprising at least one layer composed of organic compound including a light-emitting layer, sandwiched between a pair of electrodes, wherein at least one layer between the pair of electrodes contains the light-emitting material for organic light-emitting device as described in any one of 1 to 15 above.
17. A compound represented by formula (4) 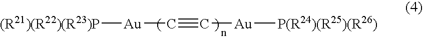
   wherein n represents an integer of 1 to 5, and R²¹ to R²⁶ independently represent each a hydrogen atom, an amino group, a cyano group, a silyl group, or an alkyl group, aryl group, alkoxy group, aryloxy group or alkylamino group that optionally has a heteroatom (provided that n is neither 1 nor 2 when R²¹ to R²⁶ all represent a cyclohexane ring).]

### DETAILED DESCRIPTION OF INVENTION

Hereinafter, the present invention will be described specifically.

The present invention provides a gold complex having a phosphorescent property which is useful as light-emitting material for an organic EL device, light-emitting material using the complex and light-emitting device using such materials. The light-emitting materials may be low molecular weight gold complex alone or polymer material obtained by polymerizing components containing the gold complexes, or composite material consisting of a light-emitting material containing the gold complex and a light-emitting material containing no gold complex.

The atomic valence of gold in the gold complex is not particularly limited, but is preferably monovalent to tetravalent, more preferably monovalent. Further, the gold complex may be an ionic complex that has a charge on the center metal. In that case, a counter ion that neutralizes the charge is present.

“Bond” as used herein refers to a chemical bond such as a covalent bond, a coordinate bond, and a dative bond. Further, “triple bond”, “single bond” and so forth represent formal bond orders.

The light-emitting material for organic light emitting device according to the present invention contains gold complexes in which gold has a bond to at least one atom selected from carbon, oxygen and sulfur. Examples of gold complex having a gold-carbon bond include alkyl complexes, alkynyl complexes, alkylidene complexes, aryl complexes, alkene complexes, alkyne complexes, carbonyl complexes, acyl complexes, cyanide complexes, isocyanide complexes, carbide complexes, and so forth. Further, examples of the gold complex in which gold has a bond to any one atom out of oxygen and sulfur include alkoxy complexes, aryloxy complexes, silyloxy complexes, carboxylate complexes, isocyanate complexes, and oxide complexes, as well as homologues of these exemplified complexes in which the oxygen atom has been replaced by a sulfur atom, and so forth. The hetero atom optionally contained in the alkyl group as described in definition of symbols in formulae (1), (2), (4), (6) and (7) is not limited as far as the atom is substituted by an alkyl group or inserted into the alkyl group, however preferred are oxygen, sulfur, nitrogen and halogen.

The nonmetallic element that can form a triple bond with the carbon atom to which gold is bonded include boron, carbon, silicon, nitrogen, phosphorus, arsenic, oxygen, sulfur, selenium, and so forth.

L¹ to L⁴ in the formulae (1), (3), and (5) represent each a monodentate or bidentate ligand and are not particularly limited so far as they can form complexes with gold. Examples thereof include phosphorus ligands (phosphine ligands, phosphite ligands, phosphide ligands and so forth), nitrogen ligands (amine ligands, pyridine ligands, nitrile ligands, phenylpyridine ligands, Schiff base ligands, and so forth), alkyl ligands, alkynyl ligands, carbonyl ligands, cyanide ligands, isocyanide ligands, diketonato ligands, carboxylato ligands, dithiocarbamato ligands, and so forth. Among these, phosphine ligands, pyridine ligands, and cyanide ligands are preferred. Also, L¹ and L², or L³ and L⁴ may be either a combination of the same ligands or a combination of different ligands.

Substituents R¹¹ to R⁵² in the respective formulae include, for example, a hydrogen atom, a halogen atom, a hydroxyl group, a nitro group, an amino group, a cyano group, a mercapto group, a silyl group, a sulfonic acid group, sulfonic acid ester groups, a phosphoric acid group, a phosphonic acid group, alkyl groups (methyl, ethyl, propyl, isopropyl, butyl, isobutyl, tertiary-butyl, amyl, hexyl, cyclopentyl groups, a cyclohexyl group and so forth), an allyl group, alkynyl groups (an ethynyl group, a propynyl group, a phenylethynyl group, a silylethynyl group, and so forth), aryl groups (a phenyl group, a naphthyl group, a biphenyl group, a vinylphenyl group, a tollyl group, and so forth), aralkyl groups (a benzyl group, a phenylethyl group, a cumyl group, and so forth), heteroaryl groups (a pyridyl group, a pyrrolyl group, an imidazolyl group, a quinolyl group, an isoquinolyl group, a thienyl group, a benzothienyl group, a furyl group, and so forth), alkoxy groups (a methoxy group, an ethoxy group, a propoxy group, an isopropoxy group, a butoxy group, an isobutoxy group, a tertiary butoxy group, and so forth), aryloxy groups (a phenoxy group, a cresolyl group, and so forth), an acetoxy group, a carboxyl group, ester groups such as an ethoxycarbonyl group, acyl groups (a formyl group, an acetyl group, and so forth), alkylamino groups, alkylthiol groups, and other organic groups. These organic groups may further have one or more substituents such as a halogen atom, a hydroxyl group, a nitro group, and an amino group. These organic groups may be bonded at one or more sites. Preferred groups among these may be different depending on the properties of the atom or atomic group to which the substituent R is bonded but is not particularly limited so far as chemical stability is not deteriorated.

In each formula, n is a parameter that greatly contributes to the color of phosphorescence and represents an integer of 1 to 5, preferably 1 to 4.

In the formulae (5) and (7), Y and Z represent organic groups that crosslink two isocyanide groups or phosphorus atoms and examples thereof include alkylene groups such as methylene, ethylene, propylene, butylenes, and hexylene, cycloalkylene groups such as a cyclohexylene group, arylene groups such as an o-phenylene group, a naphthylene group, and a ferrocenylene group, a p-menthylene group, a xylylene group, a binaphthylene group, and so forth.

In the formula (6), X⁺ represents a monovalent cation, which includes, for example, an alkali metal ion, an ammonium ion, an alkylammonium ion, a phosphonium ion, an imidazolium ion, and a pyridinium ion. Further, a single divalent cation such as an alkaline earth metal ion may be present for two gold complex ions.

The gold complexes used in the light emitting devices of the present invention can be produced using gold halide compounds as starting materials. For example, as shown in Scheme-1, the compound represented by the formula (1) can be obtained by reacting a chloro-gold complex A having a ligand L¹ with a 1-alkyn- or trimethylsilylacetylene derivative in the presence of a stoichiometric amount of a strong base (for example, sodium methoxide). In place of the strong base, a stoichiometric amount of an alkylamine (for example, triethylamine, isopropylamine, butylamine, pyrrolidine, or the like) and a catalytic amount (preferably 0.01 to 0.1 equivalent) of a copper (I) halide (for example, copper iodide, copper bromide, copper chloride) may be used. The gold complex A can be synthesized by allowing the ligand L¹ to act on gold chloride (I). As shown in Scheme-1, the compound represented by the formula (I) can also be synthesized by allowing the ligand L¹ to act on a compound B in which gold and carbon are bonded to each other. The compound B can be synthesized from gold (III) chloride by a known method (see, for example, J. Chem. Soc., p. 3220, 1962).

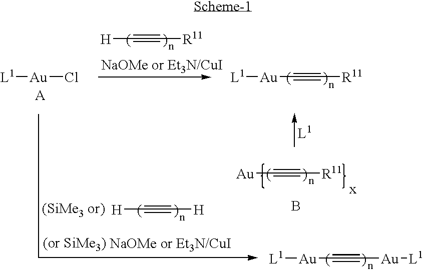

The gold complex represented by formula (2) can be produced by using a phosphorus compound as the ligand L¹ in the production method for the gold complex represented by the formula (1).

The gold complexes represented by the formula (3) or (4) can be synthesized by using, as the alkyne to be reacted with the compound A shown in the Scheme-1, 0.5 equivalent of acetylene, butadiyne, hexatriyne, octatetrayne, or decapentayne, or silylated alkynes obtained by substituting terminal hydrogens of these alkynes with silyl groups.

The compound represented by the formula (6) can be obtained by allowing 2 equivalents of a thiophenol derivative and 2 equivalents of an alkylamine to act on the gold halide complex synthesized by the known method shown in Scheme-2 (J. Chem. Soc., Dalton Trans., p. 1845, 1973).

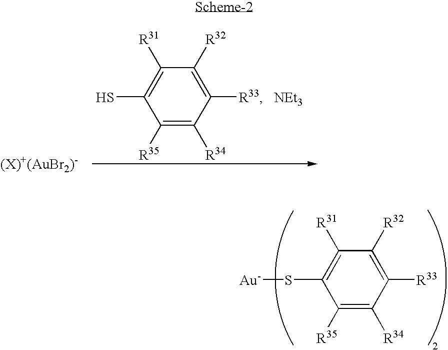

As shown in Scheme-3, the compound represented by formula (7) can be synthesized by allowing 0.5 equivalent of a phosphorus compound having a crosslinking group to act on gold (I) halide (for example, gold (I) chloride) and subsequently allowing a mercaptan compound to act thereon.

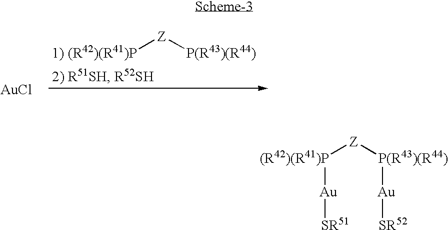

Known compounds that can be used in the light emitting devices of the present invention include, for example, gold complexes described in, for example, J. Chem. Soc., Dalton Trans., 4227 (1996), J. Am. Chem. Soc., 123, 4985, (2001), J. Chem. Soc., Chem. Commun., 243 (1989), Inorg. Chim. Acta, 197, 177 (1992), J. Chem. Soc., Dalton Trans., 3585 (2000), Inorg. Chem., 32, 2506 (1993), J. Med. Chem., 30, 2181 (1987), and so forth.

Further, gold complexes obtained by introducing a polymerizing functional group to the above-mentioned gold complexes may be polymerized to form organic polymer light emitting materials in which the gold complexes constitute a portion of the polymer.

FIG. 1 is a cross-sectional view showing one embodiment of the construction of the organic light emitting device of the present invention. In the embodiment, provided on a transparent substrate (1) are an anode (2) and a cathode (6), and provided between the anode (2) and the cathode (6) are, in order, a hole transporting layer (3), a light-emitting layer (4), and an electron transporting layer (5). The construction of the organic light emitting device of the present invention is not limited to the embodiment as shown by FIG. 1. The construction of the organic light emitting device of the present invention may be one in which either combination of (i) a hole transporting layer and a light emitting layer in order, and (ii) a light emitting layer and an electron transporting layer in order, is provided between the anode (2) and the cathode (6). Further, the construction of the organic light emitting device of the present invention may be one including only one layer, which layer contains (iii) a hole transporting material, a light-emitting material and an electron transporting material, (iv) a hole transporting material and a light-emitting material, (v) a light-emitting material and an electron transporting material, or (vi) a light-emitting material alone. Furthermore, the light-emitting layer shown in FIG. 1 is of a single layer but two or more light-emitting layers may be laminated one on another.

For film forming methods using the light-emitting material, hole transporting material and electron transporting material to constitute each layer described above, a resistance heat deposition method, an electron beam deposition method, a sputtering method, a coating method, a solution coating method, or the like can be used, but are not particularly limited thereto. In the case of low molecular weight compounds, mainly the resistance heat deposition and electron beam deposition are used while in the case of polymer materials, mainly the coating method is used in many cases.

In the organic light emitting device of the present invention, providing a hole transporting layer and an electron transporting layer on one or both sides of the light-emitting layer can achieve further improvement of light-emission efficiency and/or durability.

Examples of the hole transporting material that constitutes a hole transporting layer include known hole transporting materials, triphenylamine derivatives such as TPD (N,N′-dimethyl-N,N′-(3-methylphenyl)-1,1′-biphenyl-4,4′-diamine) and α-NPD (4,4′-bis[N-(1-naphthyl)-N-phenylamino]biphenyl), m-MTDATA (4,4′,4″-tris(3-methylphenylphenylamino)triphenylamine), polyvinylcarbazole, poly(3,4-ethylenedioxythiophene) and other known hole transporting materials can be used but are not particularly limited thereto. The hole transporting materials may be used alone or may be mixed or laminated with different hole transporting materials. The thickness of the hole transporting layer depends on the conductivity of the hole transporting layer and in not limited uniformly but is preferably 10 nm to 10 μm, more preferably 10 nm to 1 μm.

As the electron transporting material that forms the electron transporting layer, known electron transporting materials, for example, quinolinol derivative metal complexes such as Alq₃ (aluminum trisquinolinol), oxadiazole derivatives, and triazole derivatives may be used but are not particularly limited thereto. Although these electron transporting materials can be used alone, they may be mixed or laminated with different electron transporting materials. The thickness of the electron transporting layer depends on the conductivity of the hole transporting layer and is not limited uniformly but is preferably 10 nm to 10 μm, more preferably 10 nm to 1 μm.

Each of the light-emitting material, hole transporting material, and electron transporting materials as described above may be used for forming each layer, or some of the materials having different functions may be mixed to form a layer. Also, the respective layers may be formed by using polymer materials as binders. The polymer materials used for this purpose include polymethyl methacrylates, polycarbonates, polyesters, polysulfones, polyphenylene oxides, and so forth but are not particularly limited thereto.

As the anode material for the organic light emitting device of the present invention, known transparent electrode materials such as ITO (indium tin oxide), tin oxide, zinc oxide, electroconducting polymers such as polythiophene, polypyrrole, and polyaniline but are not particularly limited thereto. The surface resistance of an electrode made of such a transparent conducting material is preferably 1 to 50 Ω/□ (ohm/square). As the film forming method for these anode materials, an electron beam deposition method, a sputtering method, a chemical reaction method, a coating method and so forth can be used but are not particularly limited to these. The thickness of the anode is preferably 50 to 300 nm.

Further, between the anode and the hole transporting layer, or between the anode and an organic layer laminated adjacent to the anode, a buffer layer maybe inserted for alleviating injection barrier against hole injection. For this, a known material such as copper phthalocyanine is used but is not particularly limited thereto.

As the cathode material of the organic light emitting device of the present invention, known cathode materials, for example, Al, MgAg alloys, alkaline-earth metals such as Ca, alkali metals such as Li and Cs, alloys of Al and alkaline-earth metal such as AlCa, and alloys of alkali metal and Al such as AlLi and AlCs, may be used, but are not particularly limited thereto. As the film forming method for these cathode materials, a resistance heat deposition method, an electron beam deposition method, a sputtering method, an ion plating method, and so forth can be used but are not particularly limited to these. The thickness of the cathode is preferably 10 nm to 1 μm, more preferably 50 to 500 nm.

Further, between the cathode and an electron transporting layer, or between the cathode and an organic layer laminated adjacent to the cathode may be inserted an insulation layer having a thickness of 0.1 to 10 nm in order to increase electron injection efficiency. As the insulation layer, known materials such as lithium fluoride, magnesium fluoride, magnesium oxide, and alumina may be used but are not particularly limited thereto.

Furthermore, adjacent to the side of the light emitting layer facing toward the cathode, a hole-blocking layer may be provided in order to suppress passage of holes through the light emitting layer to recombine holes with electrons efficiently in the light emitting layer. For this, known materials such as triazole derivatives and oxadiazole derivatives are used but are not particularly limited thereto.

The substrate for the organic light emitting device of the present invention, a substrate transparent to the light-emission wavelength of the light-emitting material may be used. Known materials such as transparent plastics including glass, PET (polyethylene terephthalate), polycarbonate, and so forth may be used but are not particularly limited thereto.

The organic light emitting device of the present invention can constitute pixels of the matrix or segment type by a known method or can be used as a backlight without forming pixels.

### BRIEF DESCRIPTION OF THE DRAWINGS

FIG. 1 is a cross-sectional view showing one example of the organic light emitting device of the present invention.

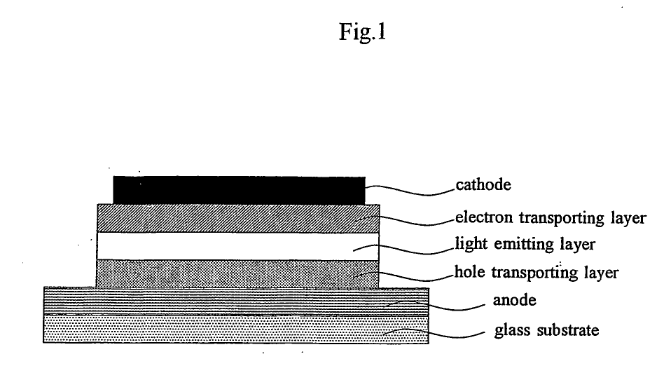

### BEST MODE FOR CARRYING OUT THE INVENTION

Hereinafter, the present invention will be described in more detail by referring to representative examples. Note that these examples are merely for the purpose of explanation and the present invention should not be considered to be limited thereto.

In the following examples, apparatuses used for analyses are as follows. Unless otherwise indicated specifically, as the reagents were used commercially available preparations (special grade) without purification.

1) ¹H-NMR
Manufactured by JEOL, Ltd., JNM EX270, 270 MHz;
Solvent: Heavy chloroform.

2) Elemental analyzer
CHNS-932 Model, Manufactured by LECO Corporation

3) GPC measurement (Molecular weight measurement)
Column: Shodex KF−G+KF804L+KF802+KF801, manufactured by SHOWA DENKO K.K.;
Eluent: Tetrahydrofuran (THF);
Temperature: 40° C.;
Detector: RI (Shodex RI-71).

4) ICP Elemental analysis
ICPS 8000, manufactured by Shimadzu Corporation

### EXAMPLE 1

### Synthesis of Phenylbutadiynyl(triphenylphosphine) Gold (I) (Compound 1-1)

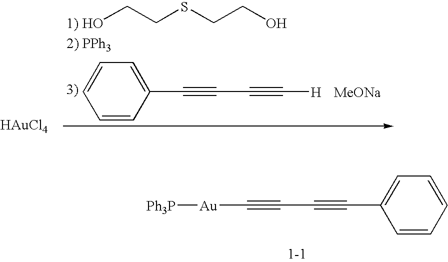

To a 10 ml ethanol solution of 0.50 g (4.1 mmol) of thiodiglycol was added 10 ml of an aqueous solution of 0.85 g (2.1 mmol) of chloroaurate (III) tetrahydrate, and the resultant mixture was stirred at room temperature for half an hour. After the reaction solution was cooled to 0° C., 0.54 g (2.1 mmol) of triphenylphosphine in 10 ml of an acetone-ethanol (volume ratio 1:1) solution was added thereto, and stirred for half an hour. The reaction solution was poured into 100 ml of water, and generated precipitate was filtered, and then dried under reduced pressure. Subsequently, 98 mg of the obtained white solids was suspended in 5 ml of methanol, to which were added 30 mg of phenylbutadiyne (0.24 mmol) and 15 mg of sodium methoxide (0.28 mmol), and the resultant mixture was stirred at room temperature for 16 hours. After the solvent was distilled off under reduced pressure from the obtained reaction mixture, a small amount of diethyl ether was added thereto, and the mixture was filtered through a glass filter and again the solvent was removed from the solution. The residue was purified by silica gel column chromatography and dried under reduced pressure to afford 65 mg (0.11 mmol) of the target compound 1-1 as pale brown solid. Identification was performed by CHN elemental analysis. The result is shown in Table 1.

### EXAMPLES 2 TO 11

Compounds 1-2 to 1-11 were synthesized in the same manner as compound 1-1 was synthesized in Example 1, by using organic phosphorous compound P(R¹⁰¹) (R¹⁰²) (R¹⁰³) in place of triphenylphosphine and by using alkyne H(C₂)ₙR¹⁰⁴ in place of phenylbutadiyne.

TABLE 1

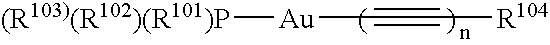

| | | | | | | | Elemental analysis(%) | |
|---|---|---|---|---|---|---|---|---|
| Example | Compound | R¹⁰¹ | R¹⁰² | R¹⁰³ | R¹⁰⁴ | n | C_{obs}(C_{calcd}) | H_{obs}(H_{calcd}) |
| 1 | 1-1 | Ph | Ph | Ph | Ph | 2 | 57.55(57.29) | 3.45(3.52) |
| 2 | 1-2 | Ph | Ph | Ph | 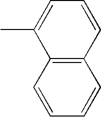 | 1 | 58.81(59.03) | 3.19(3.63) |
| 3 | 1-3 | Ph | Ph | Ph | 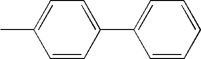 | 1 | 60.57(60.39) | 3.90(3.80) |
| 4 | 1-4 | Ph | Me | Me | o-tol | 2 | 48.34(48.12) | 3.55(3.83) |
| 5 | 1-5 | Ph | Ph | Et | 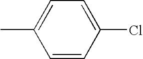 | 2 | 43.83(43.79) | 2.95(2.86) |
| 6 | 1-6 | Ph | Ph | Et | 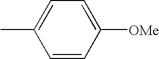 | 2 | 47.01(46.64) | 3.29(3.50) |
| 7 | 1-7 | Ph | Ph | Et | 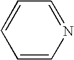 | 1 | 41.44(41.30) | 3.14(3.23) |
| 8 | 1-8 | Ph | Ph | Et | COOMe | 1 | 43.69(43.83) | 3.81(3.47) |
| 9 | 1-9 | Ph | Ph | Et | Me | 3 | 42.58(42.77) | 3.52(3.11) |
| 10 | 1-10 | EtO | EtO | EtO | t-Bu | 2 | 36.15(35.91) | 5.02(5.17) |
| 11 | 1-11 | PhO | PhO | PhO | 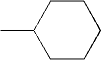 | 2 | 52.70(52.67) | 3.99(4.10) |

### EXAMPLE 12

### Synthesis of Hexanetriynediylbis(triphenylphosphine) digold (I) (Compound 2-1)

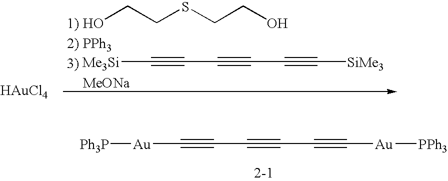

To a 10 ml ethanol solution of 0.50 g (4.1 mmol) of thiodiglycol was added 10 ml of an aqueous solution of 0.85 g (2.1 mmol) of chloroaurate (III) tetrahydrate, and the resultant mixture was stirred at room temperature for half an hour. After the reaction solution was cooled to 0° C., 0.54 g (2.1 mmol) of triphenylphosphine in 10 ml of an acetone-ethanol (volume ratio 1:1) solution was added thereto, and stirred for half an hour. The reaction solution was poured into 100 ml of water, and generated precipitate was filtered, and then dried under reduced pressure. Subsequently, 78 mg of the obtained white solids was suspended in 10 ml of methanol, to which were added 8.5 mg of sodium methoxide (0.16 mmol) and 19 mg of bis(trimethylsilyl)hexatriyne (0.087 mmol), and the resultant mixture was stirred at room temperature for 3 hours. Generated tawny precipitate was filtered through a glass filter, washed with methanol, and dried under reduced pressure to obtain 66 mg (0.067 mmol) of compound 2-1 as tawny solids. Identification was performed by CHN elemental analysis. The result is shown in Table-2.

### EXAMPLES 13 TO 15

Compounds 2-2 to 2-4 were synthesized in the same manner as compound 2-1 was synthesized in Example 12, by using organic phosphorous compound P(R¹⁰⁵)(R¹⁰⁶)(R¹⁰⁷) in place of triphenylphosphine and by using alkyne Me₃Si(C₂)ₙSiMe₃ in place of bis(trimethylsilyl)hexatriyne. Identification was performed by CHN elemental analysis. The result is shown in Table-2.

TABLE 2

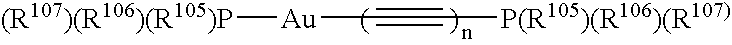

| | | | | | | Elemental analysis(%) | |
|---|---|---|---|---|---|---|---|
| Example | Compound | R¹⁰⁵ | R¹⁰⁶ | R¹⁰⁷ | n | C_{obs}(C_{calcd}) | H_{obs}(H_{calcd}) |
| 12 | 2-1 | Ph | Ph | Ph | 3 | 50.93(51.26) | 3.05(3.01) |
| 13 | 2-2 | Ph | Ph | Ph | 4 | 52.09(52.33) | 2.98(3.20) |
| 14 | 2-3 | 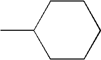 | 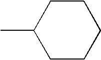 | 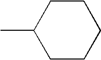 | 2 | 48.15(47.91) | 6.37(6.63) |
| 15 | 2-4 | Et | Et | Et | 3 | 30.53(30.78) | 4.50(4.31) |

### EXAMPLE 16

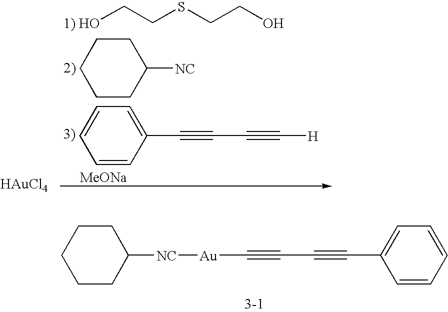

To a 10 ml ethanol solution of 0.50 g (4.1 mmol) of thiodiglycol was added 10 ml of an aqueous solution of 0.85 g (2.1 mmol) of chloroaurate (III) tetrahydrate, and the resultant mixture was stirred at room temperature for half an hour. After the reaction solution was cooled to 0° C., a solution of 0.23 g (2.1 mmol) of cyclohexylisocyanide in 10 ml of ethanol was added thereto, and stirred for half an hour. The reaction solution was poured into 100 ml of water, and generated precipitate was filtered, and then dried under reduced pressure. Subsequently, 100 mg of the obtained white solids was suspended in 10 ml of methanol, to which were added 16 mg of sodium methoxide (0.29 mmol) and 37 mg of phenylbutadiyne (0.29 mmol), and the resultant mixture was stirred at room temperature for 3 hours. Generated tawny precipitate was filtered through a glass filter, washed with methanol, and dried under reduced pressure to obtain 59 mg (0.14 mmol) of compound 3-1 as tawny solids. Identification was performed by CHN elemental analysis. The result is shown in Table-3.

### EXAMPLES 17-18

Compounds 3-2 to 3-3 were synthesized in the same manner as compound 3-1 was synthesized in Example 16, by using ligand L₅ in place of cyclohexylisocyanide and by using alkyne H(C₂)ₙR¹⁰⁸ in place of phenylbutadiyne. Identification was performed by CHN elemental analysis. The result is shown in Table-3.

TABLE 3

| | |
|---|---|
| 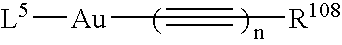 | 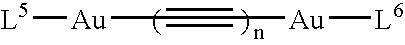 |

| | | | | | | Elemental Analysis(%) | |
|---|---|---|---|---|---|---|---|
| Example | Compound | L⁵ | L⁶ | R¹⁰⁸ | n | C_{obs}(C_{calcd}) | H_{obs}(H_{calcd}) |
| 16 | 3-1 | 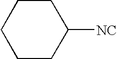 | — | Ph | 2 | 47.72(47.34) | 3.51(3.74) |
| 17 | 3-2 | 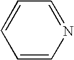 | — | Ph | 2 | 45.18(44.90) | 2.66(2.51) |
| 18 | 3-3 | 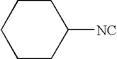 | 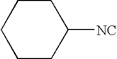 | — | 2 | 43.55(32.74) | 3.69(3.36) |

### EXAMPLES 19-20

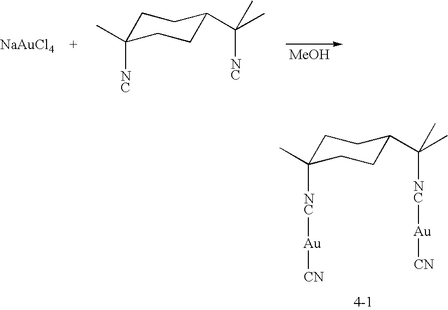

0.40 g of sodium chloroaurate (III) dihydrate (1.0 mmol) was dissolved in 10 ml of methanol, and added thereto was 0.40 g of 1,8-diisocyano-p-menthane (2.1 mmol). The resultant mixture was stirred at room temperature for half an hour, and then heated under reflux for one hour. Generated precipitate was filtered through a glass filter and cooled to −20° C. to obtain compound 4-1(Example 19).

By using 2,5-diisocyano-2,5-dimethylhexane in place of 1,8-diisocyano-p-menthane, compound 4-2 was obtained (Example 20). Identification was performed by CHN elemental analysis.

Elemental Analysis

### EXAMPLE 19

### Compound 4-1

Calcd: C, 26.43; H, 2.85; N, 8.81. Found: C, 26.80; H, 3.09; N, 9.02.

### EXAMPLE 20

### Compound 4-2

Calcd: C, 23.62; H, 2.64; N, 9.18. Found: C, 23.77; H, 2.41; N, 9.05.

### EXAMPLE 21

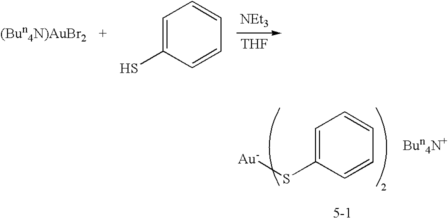

103 mg of benzenethiol (0.93 mmol) and 95 mg of triethylamine (0.93 mmol) were dissolved in 5 ml of THF and added dropwise to a 5 ml of THF solution of 279 mg (0.47 mmol) of (tetrabutylammonium) dibromo gold (I) synthesized by the known method (see P. Braunstein et al., J. Chem. Soc., Dalton Trans., 1845 (1973)). The resultant mixture was stirred at room temperature for 2 hours. After the obtained reaction mixture was filtered through a glass filter, the solvent was distilled off under reduced pressure. The oil-like residue was washed with diethyl ether, then dissolved in methanol to be recrystallized, and thereby obtained was compound 5-1. Identification was performed by CHN elemental analysis. The result is shown in Table-4.

### EXAMPLES 22-28

Compounds 5-2 to 5-8 were synthesized in the same manner as compound 5-1 was synthesized in Example 21, by using substituted benzenethiol in place of benzenethiol. Identification was performed by CHN elemental analysis. The result is shown in Table-4.

TABLE 4

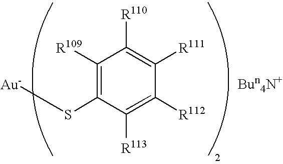

| | | | | | | | Elemental Analysis(%) | |
|---|---|---|---|---|---|---|---|---|
| Example | Compound | R¹⁰⁹ | R¹¹⁰ | R¹¹¹ | R¹¹² | R¹¹³ | C_{obs}(C_{calcd}) | H_{obs}(H_{calcd}) |
| 21 | 5-1 | H | H | H | H | H | 50.88(51.13) | 7.22(7.05) |
| 22 | 5-2 | H | Cl | H | H | H | 46.31(46.28) | 5.81(6.10) |
| 23 | 5-3 | H | Me | H | H | H | 52.22(52.54) | 7.49(7.35) |
| 24 | 5-4 | H | H | OMe | H | H | 49.89(50.20) | 6.87(7.02) |
| 25 | 5-5 | H | H | Ph | H | H | 59.60(59.31) | 6.55(6.72) |
| 26 | 5-6 | H | H | NHCOCH₃ | H | H | 49.62(49.79) | 6.91(6.79) |
| 27 | 5-7 | H | H | t-Bu | H | H | 55.83(56.16) | 8.00(8.12) |
| 28 | 5-8 | F | F | F | F | F | 39.97(40.15) | 4.58(4.33) |

### EXAMPLE 29

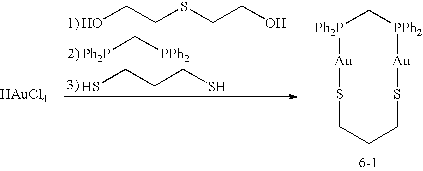

To a 10 ml ethanol solution of 0.50 g (4.1 mmol) of thiodiglycol was added 10 ml of an aqueous solution of 0.85 g (2.1 mmol) of chloroaurate (III) tetrahydrate, and the resultant mixture was stirred at room temperature for half an hour. After the reaction solution was cooled to 0° C., 0.40 g (1.1 mmol) of bis(diphenylphosphino)methane in 10 ml of an acetone-ethanol (volume ratio 1:1) solution was added thereto, and stirred for half an hour. The reaction solution was poured into 100 ml of water, and generated precipitate was filtered, and then dried under reduced pressure. Subsequently, 150 mg of the obtained white solids was suspended in methanol, to which were added 35 mg of triethylamine (0.35 mmol) and 19 mg of 1,3-propanedithiol (0.18 mmol), and the resultant was stirred at room temperature for 3 hours. Generated precipitate was filtered through a glass filter, washed with methanol, and dried under reduced pressure to obtain 89 mg of compound 6-1 as tawny solid. Identification was performed by CHN elemental analysis. The result is shown in Table-5.

### EXAMPLES 30-34

Compounds 6-2 to 6-6 were synthesized in the same manner as compound 6-1 was synthesized in Example 29, by using diphosphine (R¹¹⁴) (R¹¹⁵)P-Z-P(R¹¹⁶) (R¹¹⁷) in place of bis(diphenylphosphino)methane and by using thiol(R¹¹⁸)SH and (R¹¹⁹)SH in place of 1,3-propanedithiol. Identification was performed by CHN elemental analysis. The result is shown in Table-5.

TABLE 5

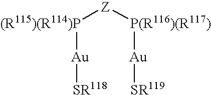

| | | | | | | Elemental analysis(%) | |
|---|---|---|---|---|---|---|---|
| Example | Compound | R¹¹⁴˜R¹¹⁷ | Z | R¹¹⁸ | R¹¹⁹ | C_{obs}(C_{calcd}) | H_{obs}(H_{calcd)} |
| 29 | 6-1 | Ph | —CH₂— | —C₃H₆— | | 37.80(38.02) | 3.47(3.19) |
| 30 | 6-2 | Ph | —C₂H₄— | Ph | Ph | 45.08(45.16) | 3.50(3.39) |
| 31 | 6-3 | Ph | —C₄H₈— | p-tol | p-tol | 46.13(46.25) | 3.91(3.69) |

| | | | | | | |
|---|---|---|---|---|---|---|
| 32 | 6-4 | Ph | 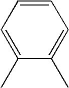 | —C₄H₈— | 43.00(42.51) | 3.49(3.36) |
| 33 | 6-5 | Ph | 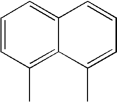 | —C₃H₆— | 44.25(44.59) | 3.63(3.24) |
| 34 | 6-6 | Me | 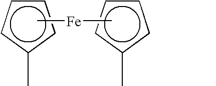 | —C₃H₆— | 25.66(25.33) | 2.96(3.25) |

### EXAMPLES 35 TO 48

### Fabrication and Evaluation of Organic Light Emitting Device

Using an ITO (indium tin oxide)-attached substrate including a 25-mm square glass substrate having formed on one side thereof two 4-mm wide ITO electrodes in the form of stripes (Nippo Electric Co., LTD.), an organic light emitting device was fabricated. First, on the ITO (anode) of the above-mentioned ITO-attached substrate was coated poly(3,4-ethylenedioxythiophene)polystyrenesulfonic acid (manufactured by Beyer AG, trade name “Vitron P” by spin coating under conditions of a revolution number of 3,500 rpm and a coating time of 40 seconds. Thereafter, drying was performed at 60° C. for 2 hours under reduced pressure in a vacuum drier to form an anode buffer layer. The thickness of the obtained anode buffer layer was about 50 nm. Then, a coating solution for forming a layer containing a light-emitting material, a hole transporting material and an electron transporting material was prepared. 8.2 μmol of a light-emitting material of the present invention, 21.0 mg (0.11 mmol) of polyvinylcarbazole as a hole transporting material, and 9.0 mg (0.025 mmol) of 2-(4-biphenyl)-5-(4-tert-butylphenyl)-1,3,4-oxadiazole (PBD) (manufactured by Tokyo Kasei Kogyo Co., Ltd.) were dissolved in 2,970 mg of chloroform (manufactured by Wako Pure Chemical Industry Co., Ltd., special grade) and the obtained solution was filtered through a filter with a hole diameter of 0.2 μm to make a coating solution. Then, this was coated on the anode buffer layer by a spin coating method under conditions of a revolution number of 3,000 rpm and a coating time of 30 seconds and dried at room temperature (25° C.) for 30 minutes to form a light emitting layer. The thickness of the obtained light emitting layer was about 100 nm. Then, the substrate on which the light emitting layer was formed was placed in a vapor deposition apparatus and calcium and aluminum were codeposited thereon at a weight ratio of 1:10 to form two cathodes arranged in the form of stripes of 3-mm wide so as to cross vertically with respect to the direction in which the anode extends. The thickness of the obtained cathode was about 50 nm. Finally, in an argon atmosphere, leadwires (wiring) were attached to the anode and cathode, respectively. Thus, four organic light emitting devices of 4 mm in length×3 mm in width were fabricated. Programmable direct current voltage/current source TR6143 manufactured by Advantest Corporation was used to apply voltage to the above-mentioned organic EL devices to cause light emission, and luminance thereof was measured using a luminance meter BM-8 manufactured by Topcon Corporation. As a result, voltage values for causing light emission and colors of the light were as shown in Table 6 (average value of four devices using respective light-emitting materials).

TABLE 6

| | | voltage | | |
|---|---|---|---|---|
| | | (V)for | | |
| | Light | causing | luminance at 15 | |
| | emitting | light | V applied | color of |
| Example | material | emission | (cd/m²) | light |
| 35 | 1-1 | 10 | 15 | blue |
| 36 | 1-2 | 10 | 220 | orange |
| 37 | 1-3 | 10 | 150 | orange |
| 38 | 1-9 | 10 | 10 | yellow |
| 39 | 2-1 | 10 | 10 | yellow |
| 40 | 2-2 | 10 | 10 | red |
| 41 | 2-4 | 10 | 5 | blue |
| 42 | 4-1 | 10 | 10 | blue |
| 43 | 5-1 | 10 | 80 | green |
| 44 | 5-2 | 10 | 50 | yellow |
| 45 | 5-3 | 10 | 20 | green |
| 46 | 5-5 | 10 | 50 | orange |
| 47 | 6-1 | 10 | 10 | green |
| 48 | 6-5 | 10 | 10 | green |

### INDUSTRIAL APPLICABILITY

With the organic light emitting device, not only visible light ranging from blue, which is shorter wavelength light, to red, which is longer wavelength light, can be emitted at low voltages, but also light emission from a triplet excited state, which has heretofore been impossible with fluorescent materials because the organic light emitting devices of the present invention utilize phosphorescent light emitting materials, so that the electric energy supplied to the device can be converted to light at high efficiencies. Further, by using either polymer compounds or low molecular weight compounds or mixtures of polymer compounds and low molecular weight compounds as materials of light emitting devices, the present invention makes it possible to easily fabricate large area devices by a coating method.

1. A material for organic light emitting device, comprising a gold complex in which gold is bonded to at least one atom selected from the group consisting of carbon, oxygen and sulfur.

2. The light-emitting material for organic light-emitting device comprising the gold complex as claimed in claim 1, wherein the at least one atom selected from the group consisting of carbon, oxygen and sulfur, bonded to gold is bonded to only one other atom than gold.

3. The light-emitting material for organic light-emitting device comprising the gold complex as claimed in claim 1, wherein the at least one atom selected from the group consisting of carbon, oxygen and sulfur, is carbon.

4. The light-emitting material for organic light-emitting device comprising the gold complex as claimed in claim 3, wherein the carbon atom bonded to gold is bonded to a nonmetallic element through a triple bond and to the gold through a single bond.

5. The light-emitting material for organic light-emitting device comprising the gold complex as claimed in claim 4, wherein the nonmetallic element is carbon.

6. The light-emitting material for organic light-emitting device as claimed in claim 5, wherein the gold complex is a compound represented by formula (1) 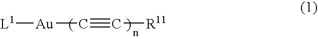
wherein L¹ represents a monodentate ligand or a bidentate ligand, n is an integer of 1 to 5, and R¹¹ represents a hydrogen atom, a halogen atom, a cyano group, a silyl group, or an alkyl group, aryl group, alkoxy group, acyl group, carboxyl group or alkoxycarbonyl group that optionally has a heteroatom.

7. The light-emitting material for organic light-emitting device as claimed in claim 6, wherein the gold complex is a compound represented by formula (2) 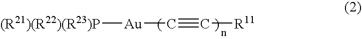
wherein n and R¹¹ have the same meanings as claimed in claim 6 above, R²¹ to R²³ independently represent each a hydrogen atom, an amino group, a cyano group, a silyl group, or an alkyl group, aryl group, alkoxy group, aryloxy group, or alkylamino group that optionally has a heteroatom.

8. The light-emitting material for organic light-emitting device as claimed in claim 5, wherein the gold complex is a compound represented by formula (3) 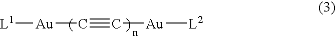
wherein L¹ and L² independently represent each a monodentate ligand or a bidentate ligand, and n represents an integer of 1 to 5.]

9. The material for organic light emitting as claimed in claim 5, wherein the gold complex is a compound represented by formula (4) 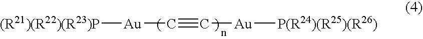
wherein n represents an integer of 1 to 5, and R²¹ to R²⁶ independently represent each a hydrogen atom, an amino group, a cyano group, a silyl group, or an alkyl group, aryl group, alkoxy group, aryloxy group or alkylamino group that optionally has a heteroatom.

10. The light-emitting material for organic light-emitting device comprising the gold complex as claimed in claim 4, wherein the nonmetallic element is nitrogen.

11. The light-emitting material for organic light-emitting device comprising the gold complex as claimed in claim 10, wherein the nitrogen atom forming a triple bond with the carbon atom bonded to gold is further bonded to another carbon atom.

12. The light-emitting material for organic light-emitting device as claimed in claim 11, wherein the gold complex is a compound represented by formula (5) 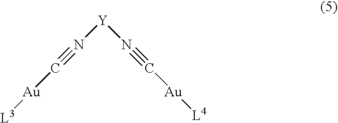
wherein Y is an alkylene, a cycloalkylene, an arylene, or an organic group consisting of two or more of the three groups, same or different, alternately bonded to each other, and L³ and L⁴ independently represent each a monodentate or bidentate ligand.

13. The light-emitting material for organic light-emitting device comprising the gold complex as claimed in claim 1, wherein the at least one atom selected from the group consisting of carbon, oxygen and sulfur is sulfur.

14. The light-emitting material for organic light-emitting device comprising the gold complex as claimed in claim 13, wherein the gold complex is a compound represented by formula (6) 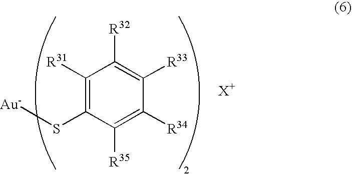
wherein R³¹ to R³⁵ independently represent each a hydrogen atom, a halogen atom, a hydroxyl group, a nitro group, an amino group, a cyano group, a mercapto group, a silyl group, a sulfonic acid group, a sulfonic acid ester group, a phosphoric acid group, a phosphonic acid group, or an alkyl group, aryl group, alkoxy group, acyl group, a carboxyl group, an alkoxycarbonyl group or acyloxy group that optionally has a heteroatom, and X⁺ represents a monovalent cation.

15. The light-emitting material for organic light-emitting device comprising the gold complex as claimed in claim 13, wherein the gold complex is a compound represented by formula (7) 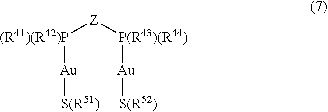
wherein R⁴¹ to R⁴⁴, R⁵¹ and R⁵² independently represent each a hydrogen atom, a cyano group, a silyl group, or an alkyl group, aryl group or acyl group that optionally has a heteroatom, and Z represents an alkylene, a cycloalkylene, an arylene, or an organic group consisting of two or more of the three groups, same or different, alternately bonded to each other.

16. An organic light emitting device, comprising at least one layer composed of organic compound including a light-emitting layer, sandwiched between a pair of electrodes, wherein at least one layer between the pair of electrodes contains the light-emitting material for organic light-emitting device as claimed in any one of claims 1 to 15 above.

17. A compound represented by formula (4) 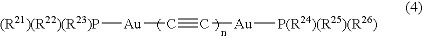
wherein n represents an integer of 1 to 5, and R²¹ to R²⁶ independently represent each a hydrogen atom, an amino group, a cyano group, a silyl group, or an alkyl group, aryl group, alkoxy group, aryloxy group or alkylamino group that optionally has a heteroatom (provided that n is neither 1 nor 2 when R²¹ to R²⁶ all represent a cyclohexane ring).
